(19) United States
(12) Patent Application Publication
(10) Pub. No.: US 2020/0253617 A1
(43) Pub. Date: Aug. 13, 2020

(54) FAST-ACTING TOURNIQUET DEVICE AND METHODS FOR USING SAME

(71) Applicant: Nathan Williams, Atlanta, GA (US)

(72) Inventor: Nathan Williams, Atlanta, GA (US)

(21) Appl. No.: 16/785,534

(22) Filed: Feb. 7, 2020

Related U.S. Application Data
(60) Provisional application No. 62/802,348, filed on Feb. 7, 2019.

(51) Int. Cl. A61B 17/135

(52) U.S. Cl. CPC A61B 17/1355; A61B 17/1322

(57) ABSTRACT

Disclosed herein are tourniquet devices and systems. A fast-acting tourniquet device may include an inflatable band with a slap band segment having a spring mechanism wherein the slap band is the medium of application of the device to a limb member; an air bladder configured to provide inflation; an air canister chamber configured to inflate the air bladder; a latch configured to secure the air bladder around the limb member; and an optional air valve configured to control release of the device. Also disclosed herein are methods of using the disclosed devices and systems, for example to control local blood flow to a limb or wound.

### REFERENCE TO RELATED APPLICATIONS

This application claims the benefit of priority to U.S. Provisional Patent Application No. 62/802,348, filed Feb. 7, 2019, which is hereby incorporated herein by reference in its entirety.

### FIELD OF DISCLOSURE

The present disclosure generally relates to one or more of the following tourniquets; namely, tourniquets with a flexible encircling member with means for applying local pressure; and methods for using same.

### BACKGROUND OF THE DISCLOSURE

In certain serious medical situations, specific medical intervention may be necessary to constrict or control blood flow to one or more limbs. For example, if blood flow is not controlled or constricted, severe blood loss from that limb can result in the loss of that limb or life. The conventional method for restricting blood flow involves using a primitive constricting or compressing device to control arterial and venous blood flow to a limb or extremity for a period of time. This often causes problems because the conventional strategy does not allow for a regulated release of pressure which can be essential in preserving the limb. Further, basic medical knowledge and training may be necessary for proper application of this medical intervention to achieve the most desirable medical outcomes. Moreover, most current medical interventions may require two hands to effectively execute. Other forms of current medical interventions also can take longer to administer than is desirable. For example, in combat situations and traumatic injuries, improper and/or delayed medical intervention may result in furthering the injury.

Accordingly, there remains a need for improved methods and devices for providing medical interventions that constrict blood flow to one or more limbs, namely, that can be self-administered using a single hand. This need and other needs are satisfied by the various aspects of the present disclosure.

### BRIEF OVERVIEW

In various aspects, the invention relates to devices, systems, and methods for restricting local blood flow, such as controlling blood flow to an injured limb. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

In accordance with the purposes of the disclosure, as embodied and broadly described herein, the disclosure, in one aspect, relates to an improved tourniquet device and system, such as, for example a quick tourniquet. In further aspects, the disclosure relates to a slap-on tourniquet. In another exemplary aspect, the disclosure relates to a fast-acting precision tourniquet. In further aspects, the disclosure also relates to an accelerated tourniquet method.

In yet another aspect, disclosed herein are devices and systems for using a fast-acting tourniquet device. In further aspects, the disclosure provides a device for controlling local blood flow, the device comprising: a pressurized gas source; a switch in operable communication with the pressurized gas source, the switch configured to selectively release or dispense a predetermined volume of gas in a gas stream having a predetermined flow rate and a predetermined flow path; and a band comprising a bendable encircling member and an air compartment [or air bladder], the compartment in operable communication with the gas stream; the encircling member having an extended state and coiled state configured to circumferentially couple around a portion of a user's body; and a fastening means configured to releasably secure the band around the portion of the user's body; wherein the gas stream is configured to fill at least a portion of the compartment effective to cause the band to exert a predetermined level of pressure against the portion of the user's body effective to reduce local blood flow.

In further aspects, the disclosure provides a fast acting tourniquet device, comprising: a slap band having a spring mechanism wherein the slap band is the medium of application of the device to a limb member; an air bladder configured to provide inflation pressure; an air canister chamber configured to trigger activation of the inflation circuit of the air bladder; a latch configured to close the air bladder around the limb member; and an air valve configured to control release of the device.

In yet another aspect, disclosed herein are methods of using the disclosed fast-acting tourniquet devices and systems. In further aspects, disclosed herein is a method, comprising: applying a slap band of a tourniquet device to a limb member; securing the tourniquet device having an air bladder around the limb member using a latch or clasp; triggering inflation circuit of the air bladder using an air canister; and controlling a release of the tourniquet device using an air valve.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 shows a depiction of a Tourniquet Device in accordance with an exemplary embodiment of the present disclosure.

FIGS. 2A-2B show depictions of a band assembly of a Tourniquet Device in accordance with an exemplary embodiment of the present disclosure.

FIGS. 3A-3C show depictions of a band assembly of a Tourniquet Device in accordance with an exemplary embodiment of the present disclosure.

FIGS. 4A-4B show depictions of a band assembly of a Tourniquet Device in accordance with an exemplary embodiment of the present disclosure.

FIG. 5 shows a depiction of an air canister assembly of a Tourniquet Device in accordance with an exemplary embodiment of the present disclosure.

FIG. 6 shows a depiction of an air canister assembly of a Tourniquet Device in accordance with an exemplary embodiment of the present disclosure.

FIG. 7 shows a depiction of an air canister assembly of a Tourniquet Device in accordance with an exemplary embodiment of the present disclosure.

FIG. 8 shows a depiction of an air canister assembly of a Tourniquet Device in accordance with an exemplary embodiment of the present disclosure.

FIGS. 9A-9B show depictions of an air canister assembly of a Tourniquet Device in accordance with an exemplary embodiment of the present disclosure.

FIGS. 10A-10B show depiction of an air canister assembly of a Tourniquet Device in accordance with an exemplary embodiment of the present disclosure.

FIG. 11 shows a depiction of a method for using a Tourniquet Device on a limb in accordance with an exemplary embodiment of the present disclosure.

FIG. 12 shows a depiction of a method for controlling blood flow to a limb using a Tourniquet Device in accordance with an exemplary embodiment of the present disclosure.

FIG. 13 is a flow chart of a method for controlling blood flow to a limb using a Tourniquet Device in accordance with an exemplary embodiment of the present disclosure.

### DETAILED DESCRIPTION OF THE DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of tourniquet devices, embodiments of the present disclosure are not limited to use only in this context. The present disclosure can be understood more readily by reference to the following detailed description of the disclosure and the Examples included therein.

Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

All publications mentioned herein are incorporated herein by reference to disclose and describe the methods and/or materials in connection with which the publications are cited.

### A. DEFINITIONS

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims, which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the disclosure. Thus, if there are a variety of additional steps that can be performed it is understood that each of these additional steps can be performed with any specific aspect or combination of aspects of the methods of the disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

The tourniquet device in this disclosure may be further referred to as a tourniquet, a tourniquet apparatus, a simple tourniquet, a quick tourniquet, a Tourinquick™, a fast-acting tourniquet, a fast action tourniquet, an instant tourniquet, and the like.

### Tourniquet Devices and Systems

Consistent with embodiments of the present disclosure, a Fast-Acting Tourniquet Device may be provided. This overview is provided to introduce a selection of concepts in a simplified form that are further described below. This overview is not intended to identify key features or essential features of the claimed subject matter. Nor is this overview intended to be used to limit the claimed subject matter's scope. The fast-acting tourniquet device may be used by individuals or medical professionals to control blood flow in medical emergencies, such as those associated with combat wounds and traumatic injuries.

In various aspects, tourniquets, such as those disclosed herein, may be a constricting or compressing device used to control arterial and/or venous blood flow to a portion of an extremity or limb for a period of time. Pressure may be applied circumferentially around a portion of a limb at a desired location. This pressure may be transferred to the walls of blood vessels, causing them to become temporarily occluded or restricted. In further aspects, the disclosed tourniquets may be configured to occlude arterial blood flow following exsanguination to produce a relatively bloodless operative field, preserve limbs, and to minimize blood loss. In emergency settings, the disclosed tourniquets may be used to stop traumatic bleeding such that medical care may be provided in time before an injured person bleeds out. In rehabilitation settings, the disclosed tourniquets may be used to restrict arterial blood flow at a consistent and/or safe pressure for short periods of time during low intensity exercise to more rapidly increase muscle size and strength.

In some instances, a primitive tourniquet may be made from a stick and a rope, leather belt, fabric, or a cloth. The rope may be made into a loop that may fit over the damaged limb and the stick may be inserted through the loop. The loop may be tightened by twisting the stick. This primitive device may stem the flow of blood but side-effects such as soft tissue damage and nerve damage may occur due to the application of unknown, uncontrollable and excessively high pressures and pressure gradients. The present disclosure provides an improved tourniquet device.

In one aspect, the genesis of the present disclosure comes from solving challenges presented by traumatic injuries in military combat situations. In such situations, applications of a tourniquet can save a life. Challenges surrounding the use of a tourniquet may include but is not limited to: improper application of a tourniquet; the amount of training it takes to learn how to properly apply a tourniquet; the time it may take for proper application; and other complexities. Such traumatic injuries are no longer just limited to combat situations. Traumatic injuries may also be part the day-to-day lives as civilians regarding incidents including but not limited to car accidents, work accidents, knifings, shootings, terrorist attacks, and other traumatic injuries.

The present disclosure therefore provides an improved tourniquet device having the ability to save lives. As most civilians lack the training and knowledge to apply a tourniquet, the present disclosure provides a wholly new and innovative tourniquet configured to allow for an extremely simple application of the disclosed tourniquet devices and apparatuses. Such simple application may be utilized by the common person to save a life. The tourniquet device and apparatus may be configured such that no previous knowledge or training may be required. Furthermore, the tourniquet device and apparatus may be configured such that it may be effective, and can be applied within seconds. The present disclosure may be configured such that it may allow a civilian to apply a tourniquet with special forces precision.

In further aspects, the present disclosure may provide a tourniquet device used to stop excessive bleeding from any limb in order to save a life. It may be used in cases of traumatic injuries like car accidents, terrorist attacks, bombings, shootings, knifings, and many other forms of trauma, including puncture wounds and various other injuries. In most of these cases, the injured actually expires due to blood loss and not by impact of the car, shrapnel, or bullet. By applying extreme pressure on and/or around a wound, the blood will stop flowing. The disclosed tourniquet is different because it can allow for easier application which may be quick to put in place, simple to use, easy enough for anyone to place, and less painful to have on while saving a life in under 10 seconds. In still further aspects, the disclosed tourniquets may be self-administered using a single hand.

Clinical literature suggests that higher tourniquet pressures are associated with higher probabilities of tourniquet-related injuries. Modern pneumatic tourniquet systems may be microcomputer-based, allowing more accurate and automatic pressure control and many important safety features to minimize the risk of patient injury. Recent trends toward personalized care has increased patient safety by allowing perioperative staff to measure and select a personalized tourniquet pressure based on the patient's Limb Occlusion Pressure. Moreover, perioperative staff may select and apply a personalized tourniquet cuff that can adapt the shape of the tourniquet cuff to a wide range of non-cylindrical limb shapes.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

According to various aspects, disclosed herein is a device for controlling local blood flow. In further aspects, the device generally comprises a pressurized gas source; a switch or mechanical barrier in operable communication with the pressurized gas source, the switch configured to selectively release or dispense a predetermined volume of gas in a gas stream having a predetermined flow rate and a predetermined flow path; and a band comprising a bendable encircling member and an air compartment or air bladder, the air compartment in operable communication with the gas stream; the encircling member having an extended state and coiled state configured to circumferentially couple around a portion of a user's body; and a fastening means configured to releasably secure the band around the portion of the user's body. In still further aspects, the gas stream may be configured to fill at least a portion of the compartment effective to cause the band to exert a predetermined level of pressure against the portion of the user's body effective to reduce local blood flow. In yet further aspects, the predetermined level of pressure is effective to stop all blood flow to the area.

In further aspects, the encircling member (or “slap band member”) may comprise a restorably deformable member having a one-way and/or directional potential energy driven state transition. In still further aspects, the encircling member can comprise a spring mechanism or means for producing a coiling action, such as a spring steel. In yet further aspects, the band may comprise a plurality of different segments or portions. The encircling member may be disposed within a first portion or segment of the band. For example, the band may comprise an assistive segment containing the encircling member or spring mechanism and a non-assistive wrapping segment without an encircling member. The band may comprise an assistive segment containing the encircling member to assist with initial clamping or encircling of the band around the body. In further aspects, after the assistive segment is slapped, clamped or otherwise secured against the body, another segment can then be wrapped around the body part and secured to the outside surface of the band using a fastener or other fastening means.

In various aspects, the encircling member is generally configured to encircle around a limb upon striking or slapping it against the limb or body part. For example, the encircling member is configured to encircle around a limb upon striking or slapping an area of the band corresponding to the location of the member against the limb. In further aspects, the encircling member may be configured to encircle around a limb upon striking or slapping it against the limb effective to cause it to go from an erect or extended state to a coiled state. In some aspects, the encircling member and/or assistive segment may comprise a thumb tab or other protrusion for guiding and/or assisting with an initial securing step.

In various aspects, the device may comprise one or more housings or casings for housing one or more device components, for example, the pressurized gas source. In further aspects, the air compartment of the band may comprise an air bladder or air chamber, or the like. The device may also include at least one gas or air supply line configured to contain and/or carry the gas stream. The gas supply line may be in fluid communication with the pressurized gas source, and/or with the air compartment, for example, to carry the gas from gas source into the air compartment for inflation. The pressurized gas source generally comprises at least one of a pressurized gas vessel, bottle, canister, cylinder, tank, and cartridge, and the like. In further aspects, the pressurized gas source may be contained within a housing, and may be detachably connected to the band.

In further aspects, the device may comprise a housing or casing for housing one or more device components. In still further aspects, the housing or casing may be configured for containing the pressurized gas source and/or container. In yet further aspects, the housing can be any shape, and preferably conforms to most hands, particularly fingers. For example, various areas may be concave or convex so to conform to areas of the body to which the device may be held. The housing can be any shape, and preferably is in the shape of a three-dimensional polygon and the housing walls define an interior space or interior sections for containing the operating elements of the invention. Any other shape (as used herein, the term shape is used in the broad sense of three-dimensional works) may be employed, so long as the shape is large enough and structured so as to be able to contain the various working components of the invention as more fully disclosed herein. The housing may be a generally hollow structure sized to contain a pressurized gas source and/or container. In yet further aspects, the device components can be all housed within the housing such that they are protected from, for example, for inadvertent shutting off or the environmental elements. In even further aspects, the housing can be comprised of plastic, plastic composite, reinforced plastic, metal, metal composite, or combinations thereof. In still further aspects, the plastic can comprise polypropylene, impact resistant plastic, or the like. The device may comprise a casing, such as band, strap, or wrap, for containing or connecting the air bladder as disclosed herein.

In further aspects, the pressurized gas source may comprise an air-tight chamber comprising a compressed gas cartridge and cartridge puncture element for releasing the pressurized gas from the gas cartridge. The chamber may be configured to contain the gas cartridge, puncture element and gas released from the gas cartridge. In still further aspects, the chamber may comprise a spring element or other means for exerting a force against an end of the gas cartridge and/or puncture element effective to cause the puncture element to release gas from the gas cartridge. In some aspects, the air chamber may comprise a housing having a plurality of portions for containing the puncture element, gas cartridge, and/or spring element.

In various aspects, the chamber may comprise removable obstructing means for mechanically obstructing the puncture element from puncturing the gas cartridge to release gas. In further aspects, the puncture element may be configured to puncture the gas cartridge to release gas upon removal of the obstructing means. To this end, the puncture element cooperates with the spring element to puncture the gas cartridge to release gas upon removal of the obstructing means. In some aspects, the pressurized gas source may be an external source remotely located from the device. In other aspects, the band may be configured to connect to external pneumatic system or air source to ease the patient out of the tourniquet instead of cutting it off directly

In further aspects, a switch or activating element may be configured to control and activate flow of gas from the pressurized gas source. The switch may be configured to control the flow of gas flowing through a gas supply line in fluid communication with the pressurized gas source. In still further aspects, the switch may be configured to control gas flow by causing a valve position to move from a closed position to an open position. In yet further aspects, the switch may be in operable communication with a puncture element for releasing pressurized gas from the pressurized gas source, for example, the switch may be operable communication with a gas cartridge puncture element for releasing pressurized gas from a gas cartridge. In some aspects, the switch may comprise a removable tab or barrier, and wherein the device may be activated by the removal of the tab or barrier. In other aspects, the switch may be a removable tab or barrier between the gas source and a puncture element for releasing pressurized gas from the gas source. In further aspects, the switch may be a removable tab or barrier between a gas cartridge and a gas cartridge puncture element for releasing pressurized gas from the gas cartridge. In yet further aspects, the switch may comprise an actuating mechanism. In still further aspects, the switch may comprise an actuating mechanism connected to a puncture element, for example, an actuating mechanism hingedly connected to a puncture element. To this end, the switch may be an actuating mechanism hingedly connected to a puncture element and is configured to activate gas release from gas source by pulling on it.

In various further aspects, the switch may comprise a toggle, lever, tab, pin, key, push-button, or the like. The switch may be in operative communication with a valve for selectively causing a valve to move from at least one of: a closed position to an open position or an open position to a closed position to control gas flow from the gas source, for example, a valve of an air cartridge. The switch can be secured to the housing at any convenient position where it may readily be actuated or pulled, and may be mechanically connected in a known manner between the pressurized gas source to control the gas dispensed into a gas stream or air bladder. As described herein, switch can be a pull tab, common on/off switch, such as a toggle, lever, push-button, or other switch. The disclosure herein contemplates any type of switch that would be practical with a pressurized gas source. As shown herein, the switch, may be a pull tab or actuating lever.

In further aspects, switch or regulator may be configured to control a flow rate of gas flowing from the pressurized gas source (or a gas supply line in fluid communication with the pressurized gas source). In still further aspects, the switch may be configured to control gas flow by moving a valve position from a closed position to an open position. In yet further aspects, the switch may comprise an actuating mechanism. In some aspects, the switch comprises a button triggered ball valve. In other aspects, the device may comprise a single valve for dispensing and/or releasing the pressurized gas. In further aspects, the switch may be in operative communication with a valve for selectively moving the valve from at least one of: a closed position to an open position and an open position to a closed position to control gas flow from gas source.

In some aspects, the device may be self-regulating. In other aspects, the device may include a gas regulator or other means for controlling the gas flow rate and/or gas volume from the gas source. In yet other aspects, the device may comprise a flow control component configured to control a gas flow rate and/or volume. The flow control component may be in fluid communication with the gas stream, and configured to control a flow rate and/or volume of a gas stream. In further aspects, the flow control component may be configured to control gas flow rate entrance into the air compartment and/or exit from the air compartment. In some aspects, the flow control component may be in fluid communication with the air compartment, and configured to control an entrance flow rate and/or entrance volume of gas. In further aspects, the flow control component may be in fluid communication with the air compartment and configured to control release of gas from the air compartment. In still further aspects, the flow control component may be in fluid communication with the air compartment, and configured to control an exit flow rate and/or exit volume of gas.

In further aspects, the placement of the pressurized gas source is variable so long as the gas flow from pressurized gas source can be dispensed so as to produce sufficient inflation and pressure for the blood flow control effects as described herein. Pressurized gas source can be placed within housing during manufacture or at any time after manufacture. In further aspects, while the pressurized gas source described herein can be permanently mounted in or on the device housing or device structure, this is not a requirement. For example, the pressurized gas source can be a cartridge configured to be removably mounted in or on a portion of the device, e.g., to allow interchange and/or replacement of the pressurized gas source. Such configurations allow users, first responders, or other medical personnel to select desired pressure profiles, and blood flow reducing-reducing properties or levels in the device, e.g., for customization purposes, for personal preferences, to match desired treatment use, a subject's physical characteristics, a subject's symptoms, or the like. To this end, the housing may use an ingress and egress element, as various embodiments of the pressurized gas source may require exchanging or replacement on occasion.

In various aspects, the pressurized gas source may comprise at least one of a pressurized gas vessel, bottle, cylinder, tank, and cartridge, and the like. In further aspects, the pressurized gas source may be contained with the device housing. In still further aspects, the pressurized gas source may be detachably connected to the device, or otherwise replaceable. In some aspects, the pressurized gas source may comprise an airtight chamber comprising with a compressed gas cartridge and cartridge puncture element for releasing the pressurized gas; wherein the chamber is configured to contain the gas cartridge, puncture element and gas released from the gas cartridge. In other aspects, the pressurized gas source may be an external gas source remotely located from the device. In further aspects, the pressurized gas source may be an external powered air compressor.

In various aspects, the fastening means or fastening component may comprise a hook and loop, hook and catch, latch, buckle, clip, snap, or the like, or any combination thereof In some aspects, fastening means is a latch, buckle, or the like. In other aspects, the fastening means or component may use a cinching mechanism for securing the band around the portion of the user's body. In further aspects, the fastening means or component may be a hook and loop arrangement. For example, the fastening means or component may be a hook connected a first end of the band and a plurality of loops disposed on an outer surface of the band.

In further aspects, the band may comprise a cuff, wrap or structural means for releasably encircling or wrapping around a portion of a user's body or limb. The band ma y be sized and dimension to releasably encircle any desired body limbs, such as a thigh or arm. To this end, the band may have a closed diameter of from about 1 in to about 36 inches, for example from about 12 inches to about 24 inches. As discussed in more detail below, the band can be comprised of neoprene or other soft material on some portions, and nylon and/or other tough and durable material on other portions.

In still further aspect, the components can be integrally or mechanically attached to other components. In a yet further aspect, the disclosed components can be connected, attached, or mounted using a connecting means, the connecting means comprising a fitting, insert, adhesive, brazing, soldering, welding, spot weld, screw with nut, rivet, fitting, insert, threading, friction fit, or snap-fit or a combination thereof In further aspects, the device components can be attached using a connecting means. In still further aspects, the connecting means can comprise a fitting, insert, adhesive, brazing, soldering, welding, spot weld, screw with nut, rivet, threading, friction fit, snap-fit, twist-lock, or interlocking mechanism or a combination thereof. In yet further aspects, the connection can be achieved using a snap, friction fitting, snap ring, O-ring, pressure fitting, clip, clasp, and the like. The snap ring or O-ring can be retained within a groove to accommodate the snap ring or O-ring. In a further aspect, the device can comprise an engagement means for coupling and holding components together. In a further aspect, the engagement means can be a screwing mechanism, a click-lock mechanism, or friction mechanism, or the like.

In further aspects, the device may comprise one or more air bladder for inflation: a band using neoprene material that is fabric backed and then stitched to a nylon strip. In other aspects, the present disclosure may comprise a band with slap band portion which may act as a medium of application to the body part. The main body of the band may comprise silicone, plastic, vinyl, nylon, cloth, cotton, or other textured fabric. In yet other aspects, the present disclosure may comprise air chamber such that it may act as a trigger for inflating the air bladder. In yet other aspects, the present disclosure may comprise a valve wherein one or more types of valves may be configured such that they may be directly coupled by EMTs and hospitals personnel to one or more devices that may control the release of pressure. In even further aspects, a tourniquet device can comprise: an inflatable band comprising a slap band segment with a spring mechanism or member; a latch or fastening means; a pressurized air source and air bladder; and an optional air valve for controlling inflation level of the air bladder.

The present disclosure may provide for civilian and professional uses. For civilian use, the tourniquet may be used in med kits or first aid kits. For professional uses, the tourniquet device may be used by EMS, paramedics, fire fighters, police officers, and for urgent care. Moreover, the tourniquet device may be configured for use within the military. Furthermore, the tourniquet device may be used in hospitals and medical centers. A tourniquet device and systems for using a fast-acting tourniquet device. A fast acting tourniquet device may be provided, comprising: a slap band having a spring mechanism wherein the slap band is the medium of application of the device to a limb member; an air bladder configured to provide an inflation circuit; an air canister combustion chamber configured to trigger activation of the inflation circuit of the air bladder; a latch configured to close the air bladder around the limb member; and an air valve configured to control release of the device.

As briefly described above, the present disclosure relates, in various aspects, to a tourniquet device. In one aspect, a tourniquet device can comprise: a band with slap portion having spring or coil mechanism, or other mechanical means for one-handedly securing the slap portion of the band around a body part by slapping band against said body part; a latch or hook, or other fastening means; an air canister chamber comprising a pressurized and air bladder; and an optional air valve for controlling inflation level and/or release of the band against the body part.

In still further aspects, the slap-band (i.e., spring or coiling) mechanism as described herein may allow for quick attachment/placement or initial securing of the band to the desired portion of the body. In yet further aspects, the user can wrap the remaining portion of the band and can use an attached latch to secure the band having the air bladder/fabric around the limb. In another aspect, the air canister chamber can then operate to inflate the air bladder and constrict local blood flow, i.e. to the limb located proximate to the secured band. In further aspects, the release of the tourniquet device may be controlled by an air valve. The air valve may be configured to allow a slow release of air, therefore not allowing the development of the potentially deadly compartment syndrome. In other aspects, once admitted to a hospital, the tourniquet device may be directly attached to a pneumatic system to regulate the control of inflation level and/or pressure of tourniquet device. In yet another aspect, the tourniquet device coupled to the pneumatic system may be configured to ease the patient out of the tourniquet instead of cutting it off directly. In further aspects, the tourniquet device may provide components which may be configured as provided in this present disclosure. For example, the slap-band (spring) mechanism, the air canister chamber, and the air bladder may be provided. In yet another aspect, an air valve couple directly from the tourniquet to a pneumatic hospital system may be provided.

In one aspect, the device may comprise a nylon strap, a buckle, a bladder, and an air canister. In a further aspect, the device may comprise a slap side, a latch having a hook and loop, an inflatable bladder having a synching action. In a yet further aspect, the device may comprise a compressed air can having a spring, a housing lower, a housing upper, a tube for airflow, a pull cord, a syringe, and a stopper or spacer. In a still further aspect, the device may comprise a retaining band, a bladder skin, a canister housing, a CO₂ canister, a pull latch, an inflatable bladder, a puncture tool, and a latch synched to a strap down. In an even further aspect, the device may comprise a strap, a buckle and pull cord. In a yet further aspect, the device may comprise an aluminum buckle at the end of tourniquet, a spring steel base, an insert into a slot or pass through loop, and thumb print for context information. In even further aspects, the device may comprise an interior touch point, stitched for latch, a hook, neoprene fabric, and a spring steel touch point.

In further aspects, the device may comprise an air cartridge which allows for inflation and snapping into place of the tourniquet device. In still further aspects, the device may comprise an interior touch point, stitched for latch, a hook, neoprene fabric, and a spring steel touch point. In yet further aspects, the device may comprise a retaining band, a bladder skin, a canister housing, a CO₂ canister, a CO₂ cartridge, a key to activate a puncture mechanism which mechanically obstructs spring-loaded puncture tool, a bold, a spring, a pin or puncture mechanism, and a CO₂ valve assembly. In yet further aspects, the device may comprise a housing A, a housing B, a housing C, a CO₂ cartridge, a spring, a valve system, a pin or key, and a puncture mechanism.

The disclosed tourniquet devices may be advantageous when it comes to both the civilian market and the professional medical market. For example, the civilian market, the present disclosure may provide for an emergency tourniquet device for first aid use. With previous tourniquets, civilians may have lacked the training required to gain the knowledge needed. For example, in the professional market, the present disclosure may provide a tourniquet device that may be quicker and more efficient than the ones currently available.

TABLE 1
Advantages and solutions provided by the present disclosure
compared with existing problems in the prior art.

| | Solutions Provided by the Present |
|---|---|
| Existing Problems In Prior Art | Disclosure |
| A tourniquet requires two hand operation of a | The slap-band spring allows for one-handed |
| tourniquet. | operation of the tourniquet device. |
| A tourniquet takes longer periods of time to put | The tourniquet device is configured to operate |
| in place. | in a short period of time. |
| Proper use of a tourniquet may require specific | The tourniquet device may be configured to |
| levels of training. Even with various levels of | utilized the slap-band spring and air cannister |
| training, applying a tourniquet may take | pull tab to allow for extremely quick application |
| anywhere from 30 seconds to 10 minutes | (For example, sub 7 seconds dominant hand, |
| | sub 10 seconds weak hand) |
| The proper application of a tourniquet may be | The tourniquet device may provide intuitive |
| extremely complicated. Most tourniquets may | directions for use that may be so simple such |
| have one or more pages of directions for proper | that the context clues on the tourniquet may |
| use. | serve as instructions. |
| Application of pressure for a tourniquet may | The tourniquet device may provide consistent |
| depend on the strength of the applier which | pressure such that it may not depend on the |
| allows for human error. Additionally, the | strength of the applier. The present disclosure |
| twisting of a metal rod, plastic piece, or | may be configured such that the constriction of |
| wooden bar with a rope may be used until | blood flow is mechanized (through the air |
| blood flow stops. This also allows for human | canister and bladder) at a pre-designated |
| error and inconsistent application of pressure. | pressure (pound per square inch) range. |
| A tourniquet may continue to be painful to put | The tourniquet device of the present disclosure |
| on/wear. | may be less painful to have on because the |
| | surface area (contact with skin) may be much |
| | larger, therefore the pressure may be spread |
| | out over a greater surface area of space. |

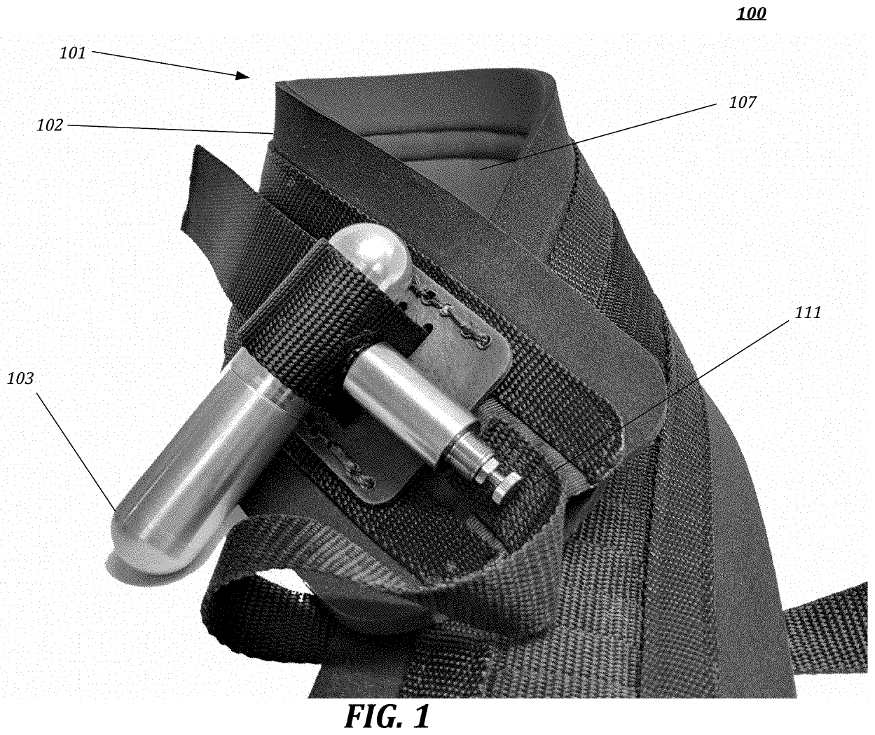

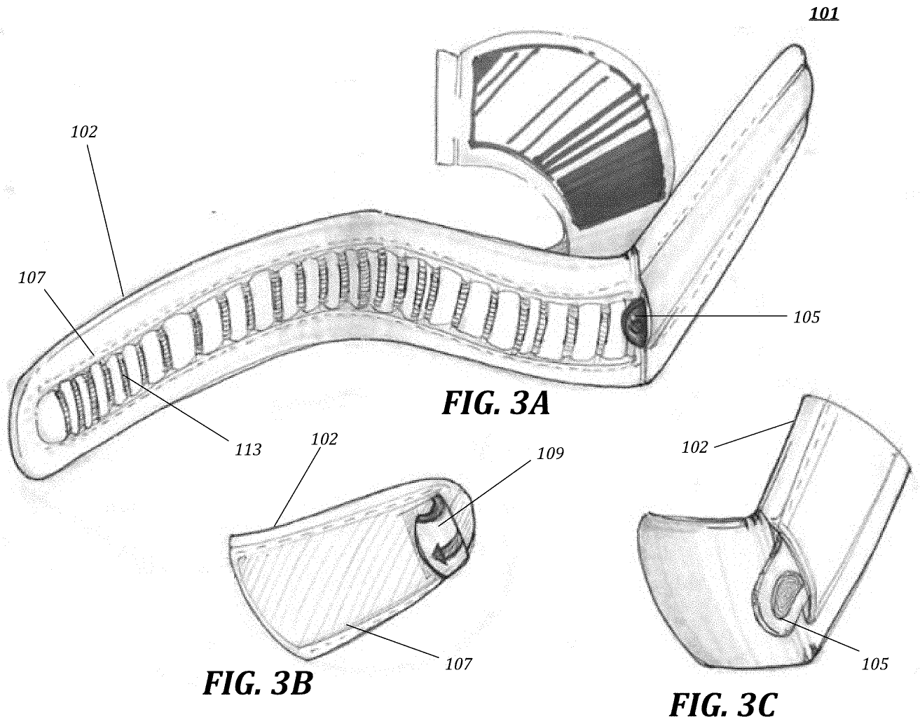

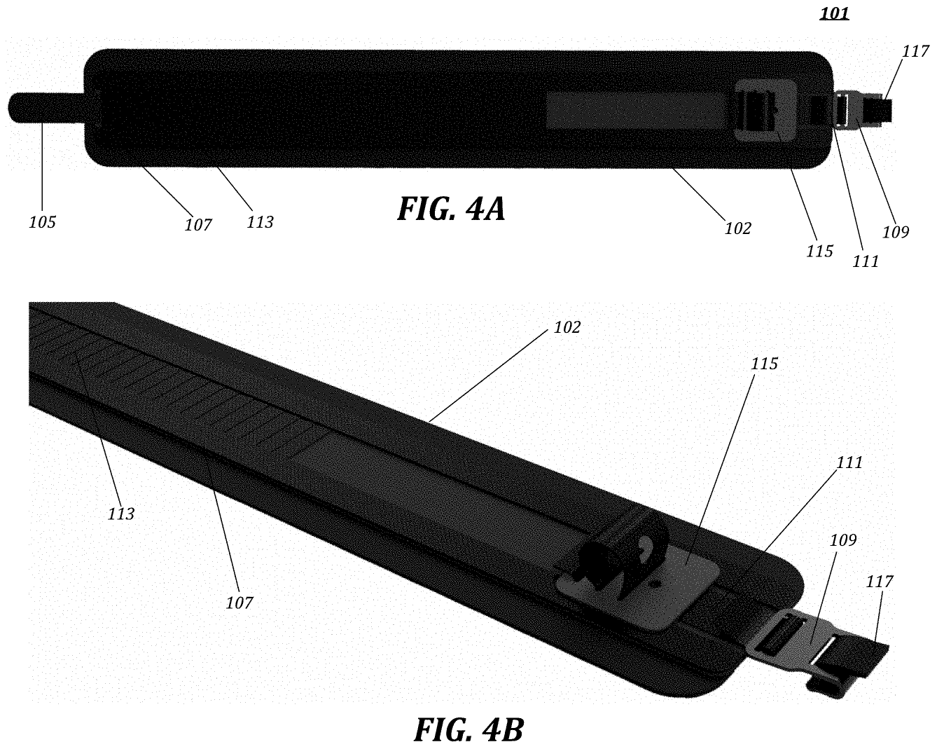

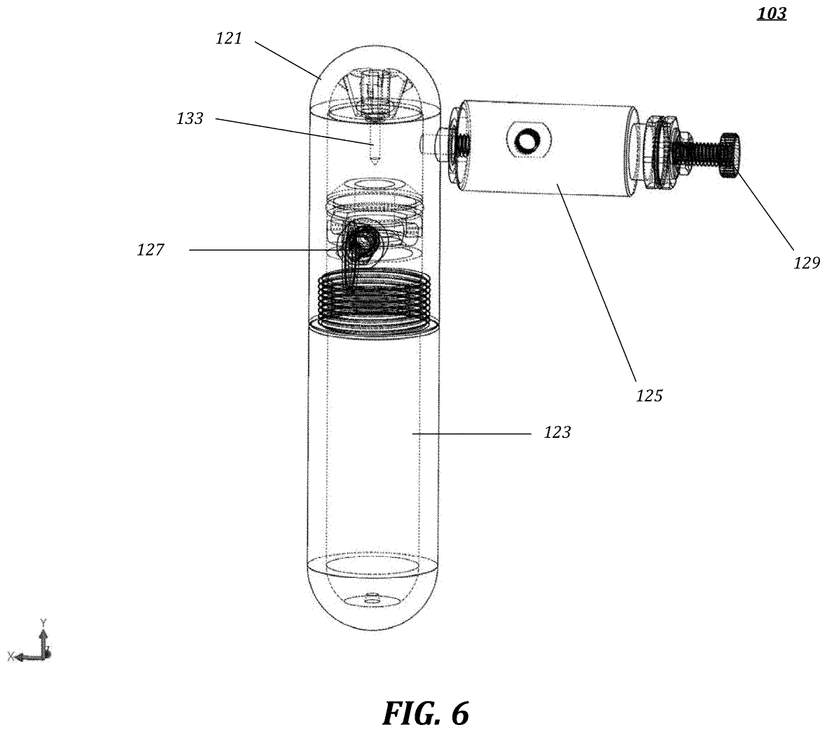

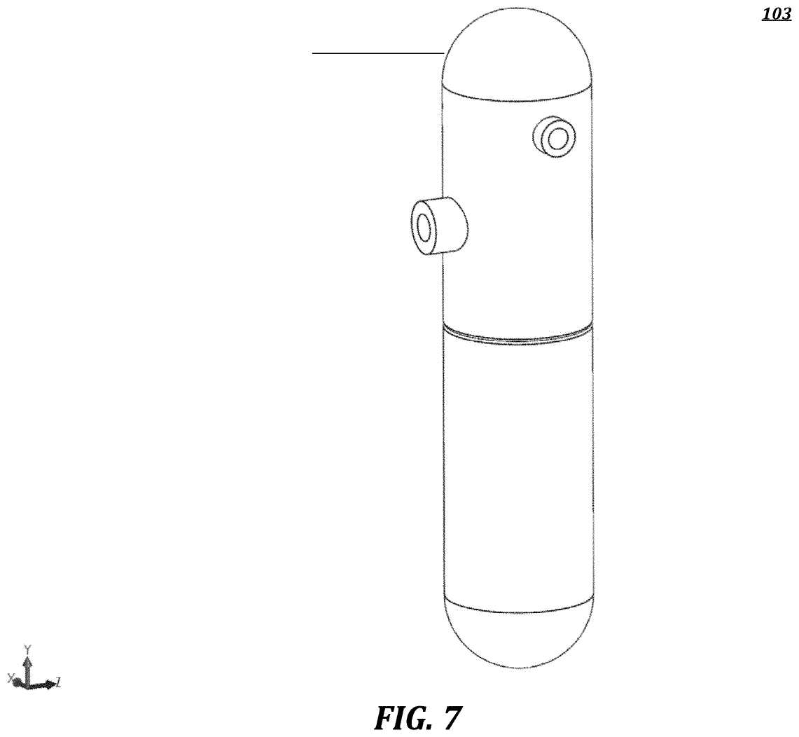

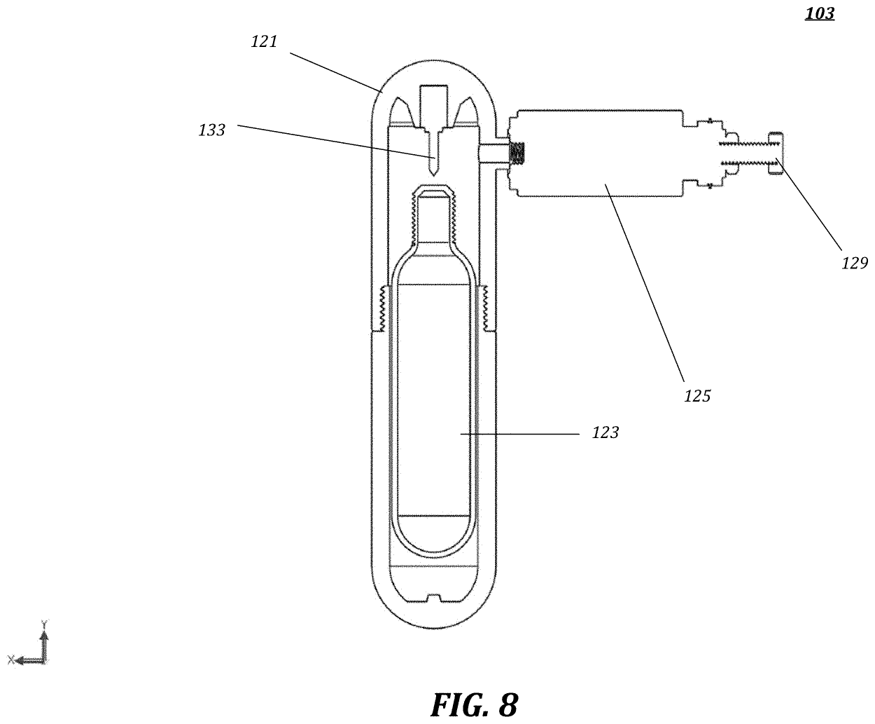

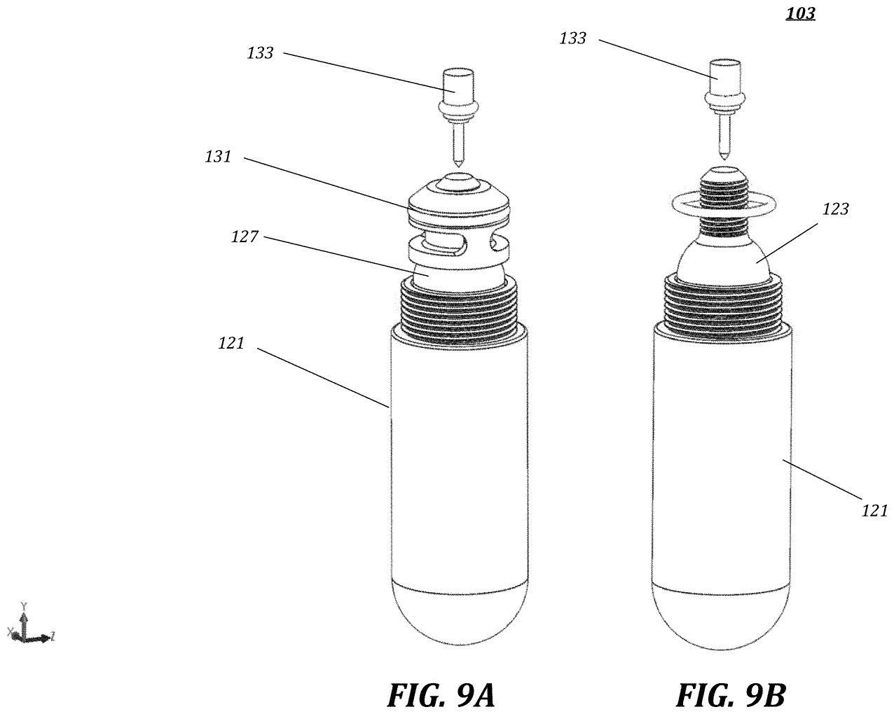

According to various further aspects of the disclosure, the tourniquet devices and systems can comprise multiple configurations. For example, various exemplary embodiments of the inventive fast acting tourniquet devices, systems, and methods are shown in FIGS. 1-13.

In one aspect, FIG. 1 depicts a device 100 for controlling blood flow to a body limb member of a user, the device comprising a band assembly 101 comprising: a band 102 with an air bladder 107 having a predetermined inflation volume and connected to the band and configured to provide a predetermined inflation level and/or inflation pressure and fastening strap 111 or other securing mechanism configured to releasably secure the band assembly and air bladder around a body limb member; and an air canister assembly 103 detachably connected to the band assembly, the air canister assembly comprising a pressurized gas source for inflating the air bladder. In an aspect, the band and air bladder cooperate to provide a medium through which pressure is applied to the body limb member.

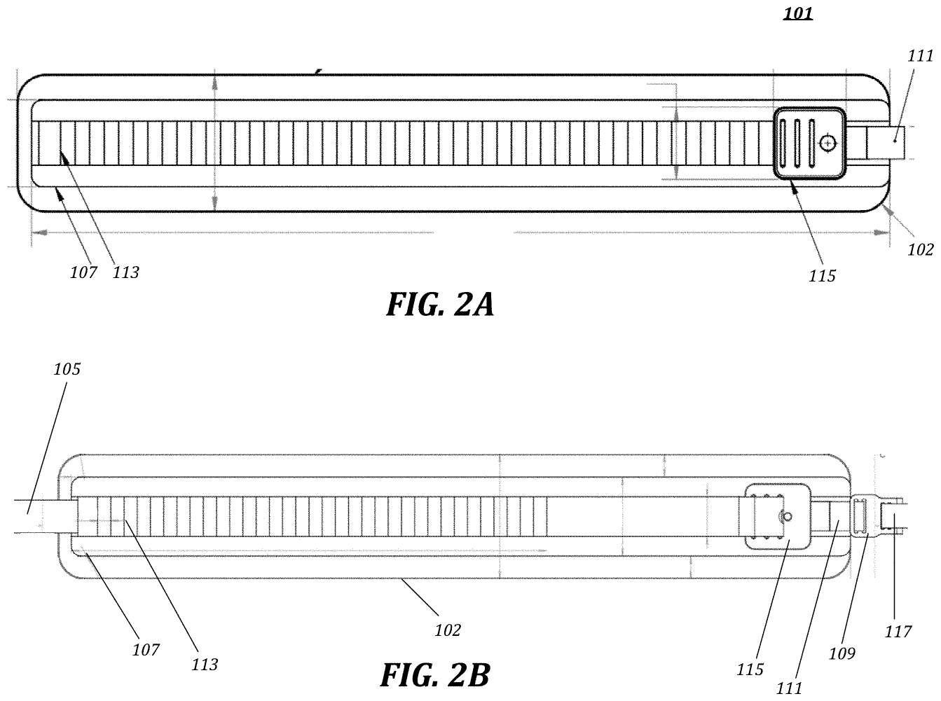

In further exemplary aspects, FIGS. 2A-4B show various depictions and components of a band assembly 101 in accordance with an embodiment of the present disclosure. As shown, band assembly includes band 102 with an assistive slap band portion including restorably deformable member 105 having a one-way and/or directional potential energy driven state transition; latch 109 connected to band using fastening strap 111. Latch 109 is configured to attach to one of the plurality of loops 113, which are formed using overlapping webbing. Latch 109 may include pull tab 117 to facilitate securing to loop 113. Band assembly 101 may further include air canister interface panel 115 for coupling air canister assembly to the band assembly. As shown, air canister interface panel 115 may include an aperture to which the pressurized gas stream is delivered to for inflating the air bladder. Air canister interface panel 115 may include a strap or other fastener for releasable securing the air canister assembly 103.

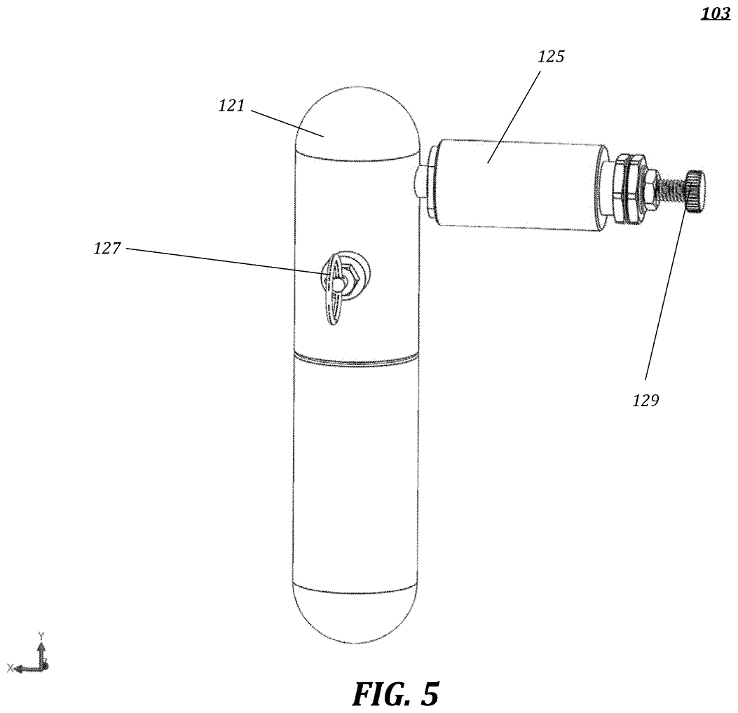

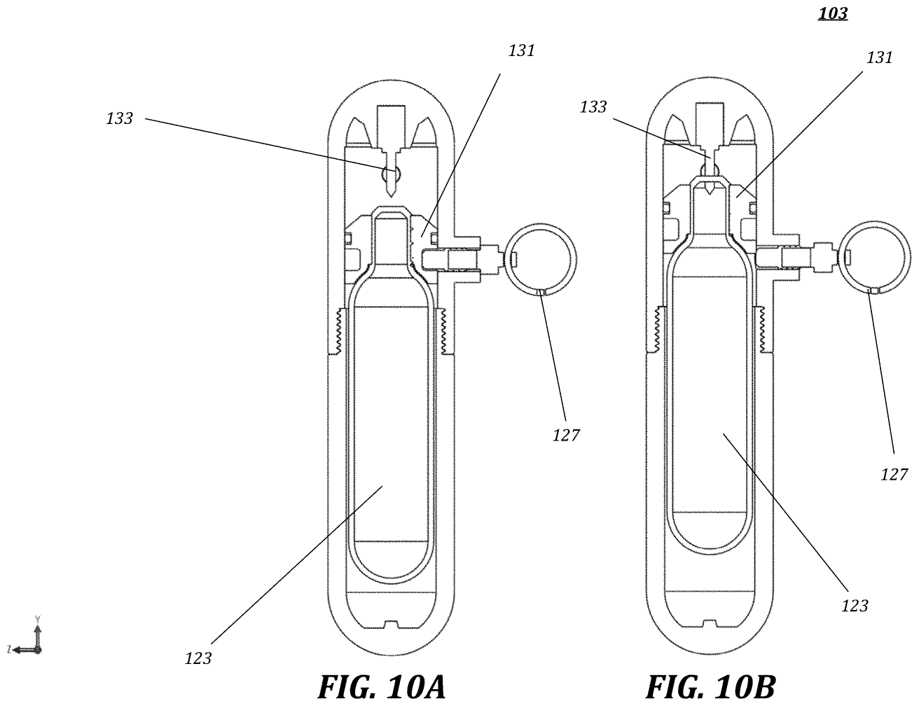

In further exemplary aspects, FIGS. 5-10B show various depictions and components of air canister assembly 103 in accordance with an embodiment of the present disclosure. Air canister assembly includes main housing 121 containing gas cartridge 123, air chamber 125 for containing pressurized air or gas therein prior to delivery to air bladder; activating pin 127 configured to activate inflation of the air bladder using the pressurized gas, and air valve with regulator 129 configured to control a rate or speed of inflation of the air bladder, or control an amount of pressure or force exerted by the device on the body limb member, or a combination thereof. Air chamber 125 includes an aperture configured to couple with the corresponding aperture on air canister assembly interface panel. As shown in FIGS. 10A-10B, removal of activating pin 127 activates tension mechanism 131, such as or spring element or other means for exerting a force against an end of the gas cartridge and/or puncture element 133, to cause the puncture element 133 to puncture gas cartridge to release gas.

The present disclosure, according to further aspects, also provides methods of using the disclosed devices and systems. In one aspect, disclosed herein is a method for using the disclosed fast-acting tourniquet devices and systems. A method may be provided for implementing a fast-acting tourniquet, comprising: applying a slap band of a tourniquet device to a limb member; securing the tourniquet device having an air bladder around the limb member using a latch; triggering inflation of the air bladder; and optionally controlling a release of the tourniquet device using an air valve. In various aspects, all the foregoing steps and methods of use may be configured to be completed using a single hand, whereby an injured user can self-administer the disclosed tourniquet devices.

In further aspects, the disclosed tourniquet devices and systems can be used to control or otherwise stop blood flow and/or bleeding. In an exemplary operation, a tourniquet may be configured to function in a particular manner. For example, initially a wound could be located. Upon identifying the wound, a user may slap on the slap band portion or segment of the inflatable band to secure an initial portion of the tourniquet device. The device uses the slap-band mechanism to initially secure the band to an area proximal to wound, such as approximately 1-6 inches or more above or below the wound. After securing the initial portion, the user can then wrap the remaining segments of the band and use the latch or fastening mean to secure in place around the limb. Subsequently thereafter, a user may pull a pull-tab to inflate an air bladder to a pre-designated pressure level or particular pounds per square inch (psi). The tourniquet device may be inflated to cause blood flow to stop, therefore not allowing the injured to bleed out. The present disclosure may be configured such that each aspect compliments another, but all may be paramount to achieve the goal of stopping the bleeding. As described herein, the slap band mechanism allows for full use and operation of the devise using a single hand, which is critical in self-administration situations where the injured in limb is a limb belonging to the user.

In operation and use, a device of the present invention is effective in achieving the methods disclosed herein. In further aspects, the pressure from the band may be applied by the device to the selected area of the subject such that the mand contacts the subject's limb. The inflated band be contacted with the surface for a time period for example, for a suitable time period so as to initiate blood flow control effects, for example, stopping blood flow to a wound or injury. The desired effect may depend on the method and technique of application of the pressure to the area. In still further aspects, after blood flow control effects are initiated, a treatment may occur to the subject or the occlusive pressure may be continued until all blood flow to the subject's limb is stopped. The occlusive pressure is applied to the subject for a time period sufficient to accomplish the effect intended in the disclosed methods, which can be between 0 seconds and several minutes or more depending on the subject and/or the method. For example, application and/or treatment time to the subject can be for a period of about 0 seconds to about 360 minutes, or longer in certain methods, to accomplish the effect intended in the disclosed methods. In further aspects, treatment times may comprise from about 0.5 minutes to about 2 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 20 minutes to about 30 minutes, from about 1 to 2 hours, from about 1 to about 3 hours, from about 2 to about 4 hours, and ranges therebetween. The treatment times listed here are not contemplated to be limiting to the methods of treatment of the invention. One of skill in the art can determine optimal treatment time periods. Also disclosed are various methods of using the disclosed devices to treat a user. For example, in another exemplary aspect, the present disclosure provides a method for controlling blood flow to a site on a patient using a disclosed device.

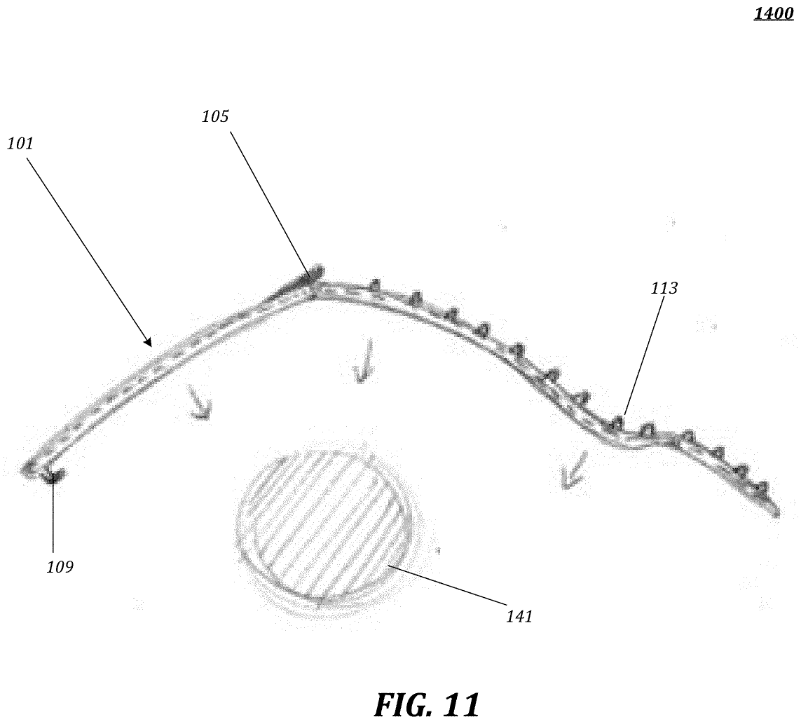

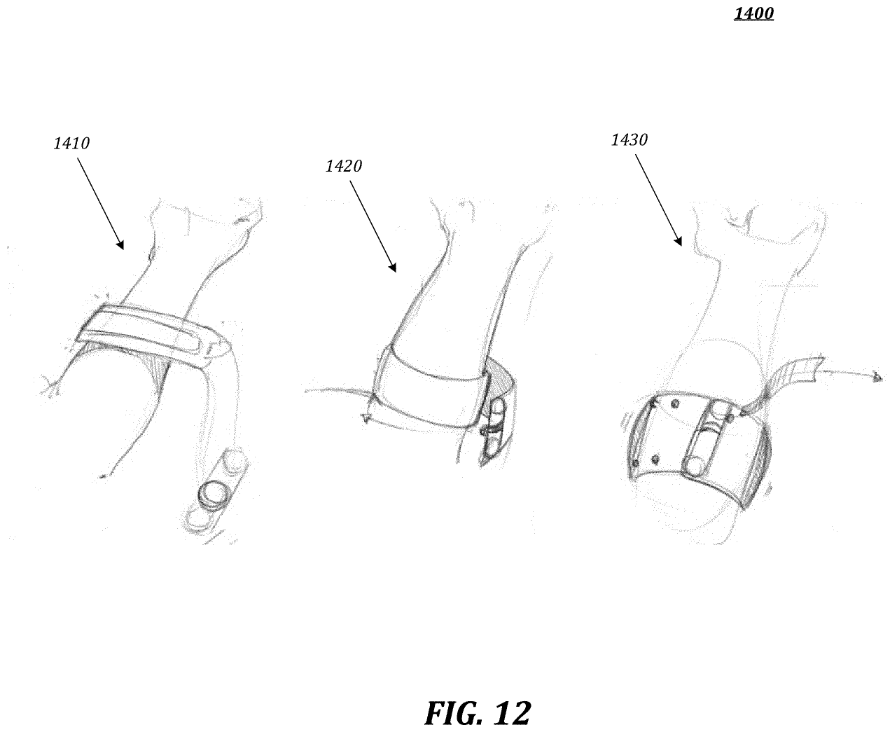

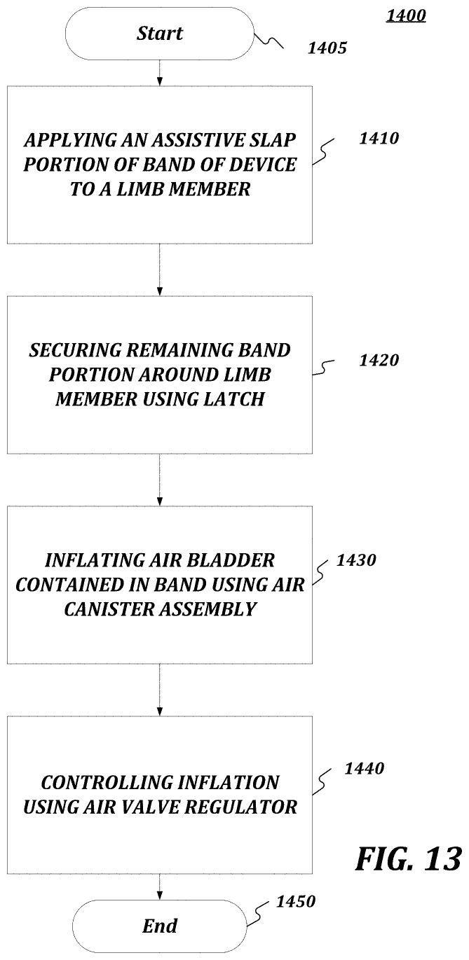

FIG. 13 show a flow chart, and FIGS. 11-12 show depictions setting forth the general stages involved in a method 1400 consistent with an embodiment of the disclosure for using a fast-acting tourniquet device, for example, to control or otherwise stop blood flow. Method 1400 may be implemented using any disclosed devices, such as, and without limitation, tourniquet device 100 as described herein. Although method 1400 has been described to be performed by tourniquet device 100, it should be understood that, in some embodiments, different operations may be performed by different elements directly coupled with tourniquet device 100. For example, a pneumatic apparatus and/or tourniquet device 100 may be employed in the performance of some or all of the stages in method 1400. Moreover, a pneumatic device may be configured much like tourniquet device 100 and, in some instances, be one and the same embodiment.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1400 will be described in greater detail below.

Method 1400 may begin at starting block 1405 and proceed to stage 1410 where a user may apply the assistive slap band segment of the inflatable band of the device to a limb member 141. For example, a paramedic or a user may apply the slap band segment to an arm or leg of a bleeding patient.

From stage 1410, where a user applied the assistive slap band segment of the device to a limb member, method 1400 may advance to stage 1420 where the remaining portion of the inflatable band can be wrapped around the limb and secured around the limb member using a latch or hook. For example, a user or a paramedic may secure the tourniquet device around an arm or leg of a bleeding patient using a pull tab attached to the latch and inserting the latch in one of a plurality of loops disposed on the outer surface of the band.

Once tourniquet device is secured around the limb member using the latch in stage 1420, method 1400 may continue to stage 1430 where the user can trigger inflation of the air bladder using an air canister. For example, a user or a paramedic may trigger inflation of the tourniquet device by activating an activating switch or pull tab connected to an air canister.

After tourniquet device has been triggered using an air canister in stage 1430, method 1400 may proceed to stage 1440 where a level and/or rate of inflation may be controlled using an air valve regulator, depending on the clinical condition and specific patient characteristics. For example, a user or paramedic may use the air valve regulator to regulate the release of pressurized gas or releasing pressure from the tourniquet device. After tourniquet device is left in place to control blood flow in 1440, method 1400 may then end at stage 1450 where the device can be removed.

The present invention includes at least the following aspects: Aspect 1: A device for controlling local blood flow in a user, the device comprising: a pressurized gas source in operable communication with a switch configured to selectively release or dispense a predetermined volume of gas from the pressurized gas source in a gas stream having a predetermined flow rate and a predetermined flow path; and a band comprising a bendable encircling member, an air compartment or air bladder compartment, and a fastening mechanism or means configured to releasably secure the band around a portion of the user's body; the air compartment in fluid communication with the gas source or gas stream; the encircling member having an extended state and coiled state, the coiled state configured to circumferentially couple around a portion of a user's body; wherein the gas stream is configured to fill at least a portion of the air compartment effective to cause the band to exert a predetermined level of pressure against the portion of the user's body effective to reduce local blood flow.

Aspect 2: A device for controlling blood flow to a body limb member of a user, the device comprising: a band assembly comprising: a band with an assistive slap band portion including a restorably deformable member having a one-way and/or directional potential energy driven state transition; an air bladder having a predetermined inflation volume and connected to the band and configured to provide a predetermined inflation level and/or inflation pressure; and a latch or other securing mechanism configured to releasably secure the band assembly and air bladder around a body limb member; an air canister assembly detachably connected to the band assembly, the air canister assembly comprising: an air canister or air chamber containing pressurized air or gas therein; an activating element configured to activate inflation of the air bladder using the pressurized gas, and an air valve configured to control a rate or speed of inflation of the air bladder, or control an amount of pressure or force exerted by the device on the body limb member, or a combination thereof; wherein the band and air bladder cooperate to provide a medium through which pressure is applied to the body limb member.

Aspect 3: The device of any preceding aspect, wherein the encircling member comprises a restorably deformable member having a one-way and/or directional potential energy driven state transition. Aspect 4: The device of any preceding aspect, wherein the encircling member comprises a spring mechanism or means for producing a coiling action, such as a spring steel.

Aspect 5: The device of any preceding aspect, wherein the encircling member is disposed within a first portion or segment of the band.

Aspect 6: The device of any preceding aspect, wherein the band comprises a plurality of different segments.

Aspect 7: The device of any preceding aspect, wherein the band comprises an assistive segment or portion containing the encircling member or spring mechanism and a wrapping segment without an encircling member.

Aspect 8: The device of any preceding aspect, wherein the band comprises an assistive segment containing the encircling member effective to assist with an initial clamping or encircling of the band around the body.

Aspect 9: The device of any preceding aspect, wherein the band comprises an assistive segment containing the encircling member effective to assist with an initial clamping or encircling of the assistive segment or portion of the band around the body.

Aspect 10: The device of any preceding aspect, wherein after the assistive segment is slapped, clamped, disposed around or otherwise secured against the body, another segment is wrapped around the body and secured to the outside surface of the band using the fastening means.

Aspect 11: The device of any preceding aspect, wherein the encircling member is configured to encircle around a limb upon striking or slapping it against the limb.

Aspect 12: The device of any preceding aspect, wherein the encircling member is configured to encircle around a limb upon striking or slapping an area of the band corresponding to the location of the member against the limb.

Aspect 13: The device of any preceding aspect, wherein the encircling member is configured to encircle around a limb upon striking or slapping it against the limb effective to cause it to go from an erect or extended state to a coiled state.

Aspect 14: The device of any preceding aspect, wherein the encircling member and/or assistive segment comprise a thumb tab or other protrusion for guiding and/or assisting with an initial securing step.

Aspect 15: The device of any preceding aspect, wherein the air compartment comprises an air bladder or chamber, or the like.

Aspect 16: The device of any preceding aspect, further comprising a housing or casing for housing one or more device components.

Aspect 17: The device of any preceding aspect, wherein the housing or casing is configured for containing a pressurized gas source or air canister.

Aspect 18: The device of any preceding aspect, further comprising at least one gas or air supply line configured to contain and/or carry the gas stream.

Aspect 19: The device of any preceding aspect, wherein the gas supply line is in fluid communication with the pressurized gas source.

Aspect 20: The device of any preceding aspect, wherein the gas supply line is in fluid communication with the air compartment or air bladder.

Aspect 21: The device of any preceding aspect, wherein the pressurized gas source comprises at least one of a pressurized gas vessel, canister, bottle, cylinder, tank, and cartridge, and the like.

Aspect 22: The device of any preceding aspect, wherein the pressurized gas source is contained with a device housing or air canister assembly.

Aspect 23: The device of any preceding aspect, wherein the pressurized gas source is detachably connected to the band or band assembly.

Aspect 24: The device of any preceding aspect, wherein the pressurized gas source comprises an air chamber comprising a compressed gas cartridge containing pressurized gas and puncture element for releasing the pressurized gas from the gas cartridge.

Aspect 25: The device of any preceding aspect, wherein an air chamber is configured to contain the gas cartridge, puncture element and gas released from the gas cartridge.

Aspect 26: The device of any preceding aspect, wherein the air chamber comprises a tension element, or spring element or other means for exerting a force against an end of the gas cartridge and/or puncture element effective to cause the puncture element to release gas from the gas cartridge.

Aspect 27: The device of any preceding aspect, wherein the air chamber comprises a housing having a plurality of portions for containing the puncture element, gas cartridge, and/or spring element.

Aspect 28: The device of any preceding aspect, wherein the chamber comprises a removable blocking element or obstructing means for mechanically obstructing the puncture element from puncturing the gas cartridge to release gas.

Aspect 29: The device of any preceding aspect, the puncture element is configured to puncture the gas cartridge to release gas upon removal of the obstructing means.

Aspect 30: The device of any preceding aspect, the puncture element cooperates with the spring element to puncture the gas cartridge to release gas upon removal of the obstructing means.

Aspect 31: The device of any preceding aspect, wherein the pressurized gas source is an external source remotely located from the device.

Aspect 32: The device of any preceding aspect, wherein the band is configured to connect to an external pneumatic system or air source effective to allow gradual device removal from user or to ease the patient out of the device instead of cutting it off directly.

Aspect 33: The device of any preceding aspect, wherein the device comprises a switch configured to control and/or activate flow of gas from the pressurized gas source.

Aspect 34: The device of any preceding aspect, wherein the switch is configured to control the flow of gas flowing through a gas supply line in fluid communication with the pressurized gas source.

Aspect 35: The device of any preceding aspect, wherein the switch is configured to control gas flow by causing a valve having a plurality of valve positions to move from a closed valve position to an open valve position.

Aspect 36: The device of any preceding aspect, wherein the valve is configured to control a rate of gas release from the pressurized gas source.

Aspect 37: The device of any preceding aspect, wherein the valve is configured to be toggled between a first rate of gas release and a second, slower rate of gas release.

Aspect 38: The device of any preceding aspect, wherein the valve is in operative communication with a toggle for changing the rate of gas release.

Aspect 39: The device of any preceding aspect, wherein the valve is in operative communication with a toggle for changing the rate of gas release between a plurality of predetermined rates of gas release.

Aspect 40: The device of any preceding aspect, wherein the valve is in operative communication with a toggle for changing the rate of gas release between a first predetermined rate of gas release and a second predetermined rate of gas release.

Aspect 41: The device of any preceding aspect, wherein the valve is in operative communication with a toggling mechanism for cooperatively controlling a rate of inflation of the air bladder.

Aspect 42: The device of any preceding aspect, wherein the valve is in operative communication with a toggling mechanism for cooperatively controlling a rate of inflation of the air bladder from a first predetermined rate of inflation to a second predetermined rate of inflation.

Aspect 43: The device of any preceding aspect, wherein the toggle or toggle mechanism is user operated for changing rates.

Aspect 44: The device of any preceding aspect, wherein a switch is in operable communication with a puncture element for releasing pressurized gas from the pressurized gas source.

Aspect 45: The device of any preceding aspect, wherein a switch is in operable communication with a gas cartridge puncture element for releasing pressurized gas from a gas cartridge.

Aspect 46: The device of any preceding aspect, wherein the switch comprises a removable tab or barrier, and wherein the device may be activated by the removal of the tab or barrier.

Aspect 47: The device of any preceding aspect, wherein the switch comprises a removable tab or barrier between the gas source and a puncture element for releasing pressurized gas from the gas source.

Aspect 48: The device of any preceding aspect, wherein the switch comprises a removable tab or barrier between a gas cartridge and a gas cartridge puncture element for releasing pressurized gas from the gas cartridge.

Aspect 49: The device of any preceding aspect, wherein the switch comprises an actuating mechanism.

Aspect 50: The device of any preceding aspect, wherein the switch comprises an actuating mechanism connected to a puncture element.

Aspect 51: The device of any preceding aspect, wherein the switch comprises an actuating mechanism hingedly connected to a puncture element.

Aspect 52: The device of any preceding aspect, wherein the switch comprises an actuating mechanism hingedly connected to a puncture element and is configured to activate gas release from gas source by pulling on the switch.

Aspect 53: The device of any preceding aspect, wherein the switch comprises a toggle, lever, tab, pin, key, push-button, button-triggered valve, or the like.

Aspect 54: The device of any preceding aspect, wherein the switch is in operative communication with a valve for selectively causing a valve to move from at least one of: a closed position to an open position or an open position to a closed position to control gas flow from the gas source.

Aspect 55: The device of any preceding aspect, wherein the device is self-regulating.

Aspect 56: The device of any preceding aspect, further comprising a gas regulator or other means for controlling a gas flow rate and/or gas volume dispensed from the gas source.

Aspect 57: The device of any preceding aspect, further comprising a flow control component configured to control a gas flow rate and/or gas volume.

Aspect 58: The device of any preceding aspect, further comprising a flow control component in fluid communication with the gas stream, and configured to control a gas flow rate and/or gas volume from a gas stream.

Aspect 59: The device of any preceding aspect, wherein the flow control component is configured to control gas flow rate and/or volume entrance into the air compartment and/or exit from the air compartment.

Aspect 60: The device of any preceding aspect, further comprising a flow control component in fluid communication with the air compartment, and configured to control an entrance flow rate of gas and/or an entrance volume of gas.

Aspect 61: The device of any preceding aspect, further comprising a flow control component in fluid communication with the air compartment and configured to control release of gas from the air compartment.

Aspect 62: The device of any preceding aspect, further comprising a flow control component in fluid communication with the air compartment, and configured to control an exit flow rate and/or exit volume of gas.

Aspect 63: The device of any preceding aspect, wherein the fastening component or means comprises a hook and loop, hook and catch, latch, buckle, clip, snap, or the like.

Aspect 64: The device of any preceding aspect, wherein the fastening component or means is a latch, buckle, or the like.

Aspect 65: The device of any preceding aspect, wherein the fastening component or means comprises a cinching mechanism for securing the band around the portion of the user's body.

Aspect 66: The device of any preceding aspect, wherein the fastening component or means is a hook and loop arrangement.

Aspect 67: The device of any preceding aspect, wherein the fastening component or means is a hook connected a first end of the band and a plurality of loops disposed on an outer surface of the band.

Aspect 68: The device of any preceding aspect, wherein the band comprises a cuff, wrap or structural means for encircling a portion of a user's body or limb.

Aspect 69: The device of any preceding aspect, wherein the band is sized and dimensioned effective to releasably encircle a body limb, such as a thigh or arm.

Aspect 70: The device of any preceding aspect, wherein the band has a closed diameter of from about 1 in to about 36 inches, for example from about 12 inches to about 24 inches.

Aspect 71: The device of any preceding aspect, wherein the band has an opened length of from about 12 in to about 48 inches, for example from about 12 inches to about 36 inches.

Aspect 72: The device of any preceding aspect, wherein the band is comprised of neoprene or other soft material, or nylon and/or other tough and durable material, or combinations thereof.

Aspect 73: The device of any preceding aspect, wherein the valve comprises an air valve configured to control an inflation level of the air bladder by allowing a controlled release of gas or air, wherein the controlled release is at least one of consistent, intermittent, and variable.

Aspect 74: The device of any preceding aspect, wherein the air valve is further configured to control release of the device by allowing a controlled release of gas or air, wherein the controlled release is at least one of consistent, intermittent, and variable.

Aspect 75: The device of any preceding aspect, wherein the air valve is further configured to be directly attached to a pneumatic system wherein the direct attachment may control inflation level, such as by gradual deflation/inflation or gas/air release.

Aspect 76: A method for controlling local blood flow to a body limb or body part of a user, the method comprising the steps of: positioning a deformable slap band of a tourniquet device having an air bladder on and/or around a body limb or body part; securing the tourniquet device to the body limb or part using a fastening component or latch; and inflating the air bladder using pressurized gas contained within an air canister or chamber effective to control blood flow local to the body part or body limb.

Aspect 77: The method of any preceding aspect, further comprising controlling a rate of inflation or an inflation level of the air bladder using an air valve in fluid communication with the air bladder.

Aspect 78: The method of any preceding aspect, wherein all the method steps can be performed by a single hand of a user.

Aspect 79: The method of any preceding aspect, wherein all the method steps can be performed by a user on themselves using a single hand.

Aspect 80: The method of any preceding aspect, wherein control of local blood flow comprises complete block of blood flow.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order.

1.-75. (canceled)

76. A method for controlling local blood flow to a body limb or body part of a user, the method comprising the steps of:
positioning a deformable slap band of a tourniquet device having an air bladder on and/or around a body limb or body part;
securing the tourniquet device to the body limb or part using a fastening component or latch; and
inflating the air bladder using pressurized gas contained within an air canister or chamber effective to control blood flow local to the body part or body limb.

77. The method of claim 76, further comprising controlling a rate of inflation or an inflation level of the air bladder using an air valve in fluid communication with the air bladder.

78. The method of claim 77, wherein all the method steps can be performed by a single hand of a user.

79. The method of claim 78, wherein all the method steps can be performed by a user on themselves using a single hand.

80. The method of claim 79, wherein control of local blood flow comprises complete block of blood flow.

81. A device for controlling local blood flow in a user, the device comprising:
a pressurized gas source in operable communication with a switch configured to selectively release a predetermined volume of gas from the pressurized gas source in a gas stream having a predetermined flow rate and a predetermined flow path; and
a band comprising a bendable encircling member, an air compartment, and a fastening mechanism configured to releasably secure the band around a portion of the user's body; the air compartment in fluid communication with the gas source; the encircling member having an extended state and coiled state, the coiled state configured to circumferentially couple around a portion of a user's body;
wherein the gas stream is configured to fill at least a portion of the air compartment effective to cause the band to exert a predetermined level of pressure against the portion of the user's body effective to reduce local blood flow.

82. The device of claim 81, wherein the encircling member comprises a restorably deformable member having a one-way or directional potential energy driven state transition.

83. The device of claim 82, wherein the encircling member comprises a spring mechanism for producing a coiling action.

84. The device of claim 82, wherein the band comprises an assistive segment containing the encircling member and a wrapping segment without an encircling member.

85. The device of claim 84, wherein after the assistive segment is secured against a portion of the body, the wrapping segment is wrapped around said portion the body and secured to an outside surface of the band using the fastening mechanism.

86. The device of claim 85, wherein the pressurized gas source is contained within an air canister assembly.

87. The device of claim 86, wherein the pressurized gas source is detachably connected to the band.

88. The device of claim 87, wherein the pressurized gas source comprises an air chamber comprising a compressed gas cartridge containing pressurized gas and puncture element for releasing the pressurized gas from the gas cartridge.

89. The device of claim 87, wherein the band is configured to connect to an external pneumatic system or air source effective to allow gradual device removal from user.

90. The device of claim 87, wherein the device comprises a switch configured to activate flow of gas from the pressurized gas source.

91. The device of claim 90, further comprising a flow control component in fluid communication with the gas source, and configured to control a gas flow rate or gas volume from a gas stream, or combination thereof.

92. The device of claim 91, wherein the flow control component is configured to control gas flow rate or volume entrance into the air compartment or exit from the air compartment, or combinations thereof.

93. A device for controlling blood flow to a body limb member of a user, the device comprising:
a band assembly comprising:
a band with an assistive slap band portion including a restorably deformable encircling member having a one-way directional potential energy driven state transition;
an air bladder having a predetermined inflation volume and connected to the band and configured to provide a predetermined inflation level or inflation pressure; and
a fastening mechanism configured to releasably secure the band assembly and air bladder around a body limb member
an air canister assembly detachably connected to the band assembly, the air canister assembly comprising:
an air canister containing pressurized gas therein;
an activating switch configured to activate inflation of the air bladder using the pressurized gas, and
an air valve configured to control a rate of inflation of the air bladder, or control an amount of pressure or force exerted by the device on the body limb member, or a combination thereof;
wherein the band and air bladder cooperate to provide a medium through which pressure is applied to the body limb member effective to reduce local blood flow to said body limb member.

94. The device of claim 93, wherein the fastening mechanism comprises a hook connected to a first end of the band and a plurality of loops disposed on an outer surface of the band.

95. The device of claim 94, wherein the band comprises a cuff or wrap configured for encircling a portion of a user's body limb member.
